(19) United States
(12) Patent Application Publication
(10) Pub. No.: US 2018/0117157 A1
(43) Pub. Date: May 3, 2018

(54) STABLE PHARMACEUTICAL COMPOSITION, COMPRISING AN AQUEOUS SOLUTION OF AN ANTIBODY-DERIVED THERAPEUTICALLY ACTIVE PROTEIN

(71) Applicant: ADOCIA, Lyon (FR)

(72) Inventors: Olivier SOULA, Meyzieu (FR); Thomas BALLET, Lyon (FR)

(73) Assignee: ADOCIA, Lyon (FR)

(21) Appl. No.: 15/854,434

(22) Filed: Dec. 26, 2017

Related U.S. Application Data
(63) Continuation of application No. 14/428,860, filed on Mar. 17, 2015, now abandoned.
(60) Provisional application No. 61/702,522, filed on Sep. 18, 2012.

(51) Int. Cl. A61K 47/18; C07K 16/28; C07K 14/705; C07K 16/22; C07K 16/24; A61K 9/00; A61K 9/08

(52) U.S. Cl. CPC A61K 47/186; C07K 16/2887; C07K 14/70521; C07K 16/22; C07K 16/241; C07K 16/2863; C07K 2319/30; C07K 2317/24; C07K 2317/52; C07K 2317/569; C07K 2317/94; A61K 9/08; A61K 9/0019

(57) ABSTRACT

A storage-stable pharmaceutical composition includes an aqueous solution of at least an antibody-derived therapeutically active protein chosen amongst antibody, nanobody or fusion protein and an amount effective to stabilize the antibody-derived therapeutically active protein of at least one lauryldimethylamineoxide and/or of one of its amine oxide analogs.

The present invention relates generally to compositions and methods thereof that increase physical stability by reducing or preventing aggregation of antibody-derived proteins in therapeutically useful formulations, and specifically, to compositions having at least an antibody-derived therapeutic protein and at least lauryldimethylamineoxide and/or one of its alkylamine oxide analogs.

Like most of protein products, antibody-derived therapeutics proteins are known to be unstable in liquid formulations. Indeed, proteins undergo numerous physical and chemical changes that affect potency and safety. Among these are aggregation, which includes formation of dimers, trimers, and higher-order protein aggregates as described in Mahler, H. C. et al. Induction and Analysis of Aggregates in a Liquid IgG1-Antibody Formulation. Eur.J.Pharm.Biopharm. 2005, 59 (3), 407-417 or in Mahler, H. C. et al. Protein Aggregation: Pathways, Induction Factors and Analysis. 3 Pharm.Sci. 2009, 98 (9), 2909-2934.

Aggregation may occur during the course of manufacturing process or during storage because of high-shear and mechanical stresses. Furthermore, the delivery of high protein concentration is often required for parenteral administration, especially for subcutaneous injection, due to dose requirements (usually ≥50 mg/mL, preferably ≥100 mg/mL) and to the volume limitations (≤1.5 mL). Such concentrated protein solutions generate several problems, including the tendancy of proteins to aggregate during processing and/or storage, because of the increased likelihood of protein-protein interactions in concentrated regimen.

In an attempt to obviate the physical stability problem, protein formulations have been formulated and commercialized in a dry form, i.e. in a lyophilized form. For example, stable lyophilized protein formulations are disclosed in PCT publication WO 97/04801. The disclosed lyophilized formulations can be reconstituted to generate high protein-concentration liquid formulations without apparent loss of stability. However, the convenience of administration and improved patient compliance make a stable liquid formulation a better choice than a lyophilized product.

In the case of liquid formulations, the physical stability problem is usually circumvented by adding a suitable surfactant, most of the time polysorbate 20 or polysorbate 80. Polysorbates are the most widely used non-ionic surfactants to stabilize protein pharmaceuticals against interface-induced aggregation and surface adsorption.

They have been shown to be quite effective against various stresses such as agitation, freeze/thawing and lyophilization. Nevertheless, some of their characteristics need to be carefully considered and monitored. Indeed, commercially available polysorbates 20 and 80 are chemically diverse mixtures containing mainly sorbitan polyoxyethylene (POE) fatty acid esters.

Additionally, substantial amounts of POE, sorbitan POE and isosorbide POE fatty acid esters are present. This leads to a significant degree of lot-to-lot variability requiring a close scrutiny of each lot in order to ensure uniform behavior. The presence of residual levels of peroxide in bulk polysorbate is also a concern.

The European Pharmacopeia specifies a limit for peroxide number ≤10. There have been reports of a buildup of peroxides in bulk as well as in aqueous solutions of polysorbate, when exposed to ambient oxygen and light.

Depending on handling and storage conditions and supplier lot, varying concentrations of peroxides (9 ppm-250 ppm) were noted among different lots of polysorbates (See for example Kishore, R. S. et al. Degradation of Polysorbates 20 and 80: Studies on Thermal Autoxidation and Hydrolysis. J Pharm.Sci. 2010. or Kishore, R. S. et al. The Degradation of Polysorbates 20 and 80 and Its Potential Impact on the Stability of Biotherapeutics. Pharm.Res 2011, 28 (5), 1194-1210).

The buildup of peroxides can be detrimental not only to the stability of polysorbate itself but also to the therapeutic protein, which it stabilizes. For example, it has been described in the literature that certain polysorbate concentrations result in increased protein aggregation (See Kiese, S. et al. Shaken, Not Stirred: Mechanical Stress Testing of an IgG1 Antibody. J Pharm.Sci. 2008, 97 (10), 4347-4366).

There is thus a need of stabilizing agents which solve totally or in part the problems discussed above.

It is known from Lee et al. that lauryldimethylamineoxide can be used to reconstitute an integral membrane protein such as CD40, that is not antibody-derived therapeutically active protein, into a phosphatidyl-choline liposome. According to the method of reconstitution described, lauryldimethylamineoxide is removed by dialysis during the process of reconstitution. The reconstituted solutions are then mixed and incubated with CD40 antibody in order to confirm by immunogold labeling the incorporation of CD40 into the liposomal membrane.

Patent application EP1816460 discloses a method of stabilizing analytes (nucleic acid or (poly)peptide molecules). Detergents (denaturing agents) are known for solubilizing proteins. According to EP1816460, detergents may be present during immunoassay, as said immunoassay is tolerant against certain concentrations of detergent. Among the many detergents listed in this patent application, some of them, such as Polysorbate 20 (Tween) for instance, have been proven ineffective to stabilize a therapeutically effective proteins/peptides such as an antibody, a nanobody or a fusion protein. The one skilled in the art knows moreover that the activity of denatured proteins is decreased or lost.

From WO2009/081136 A1, is known a method of producing a conformational specific binder partner of a G-protein coupled receptor wherein the so-called mutant GPCR has increased stability compared to the parent GPCR. The binding partner is necessary to stabilize the GPCR during cristallisation procedures since harsh detergents such as DDM, C₈ to C₁₁ maltoside or glucoside, lauryldimethylamineoxide and SDS are used.

These documents do not describe the use of lauryldimethylamineoxide and/or of one of its amine oxide analogs to stabilize an antibody-derived therapeutically active protein in aqueous solutions.

The present invention relates to a method to formulate storage-stable pharmaceutical compositions, comprising an aqueous solution of a therapeutically effective proteins/peptides such as an antibody, a nanobody or a fusion protein-based on the surprising discovery that lauryldimethylamineoxide and/or of one of its amine oxide analogs had the ability to stabilize aqueous solutions of therapeutically effective proteins/peptides.

The present invention relates to the use of one lauryldimethylamineoxide and/or of one of its amine oxide analogs to stabilize an antibody-derived therapeutically active protein in aqueous solutions and to the storage-stable pharmaceutical compositions obtained.

The invention also relates to the use of at least one lauryldimethylamineoxide and/or of one of its amine oxide analogs to stabilize a stored aqueous pharmaceutical formulation of an antibody-derived therapeutically active protein such as an antibody, a nanobody or a fusion protein.

A concentration expressed in M is a concentration in mol/L.

A concentration expressed in mM is a concentration in mmol/L.

Surprisingly, the aqueous solution of a therapeutically effective proteins/peptides is better stabilized by adding at least one lauryldimethylamineoxide and/or of one of its amine oxide analogs according to the present invention than by adding a polysorbate.

In particular the stabilizing effect obtained is independent from the pH of the aqueous solution of the therapeutically effective proteins/peptides, more particularly within at least one of the pH range as defined below.

In a further embodiment, the aqueous solution has a pH that is in the range from 5.0 to 8.0.

In a further embodiment, the aqueous solution has a pH that is in the range from 5.5 to 7.8.

In a further embodiment, the aqueous solution has a pH that is in the range from 5.0 to 6.5.

In a further embodiment, the aqueous solution has a pH that is in the range from 5.5 to 6.5.

In a further embodiment, the aqueous solution has a pH that is in the range from 6.0 to 8.

In a further embodiment, the aqueous solution has a pH that is in the range from 6.0 to 7.5.

In a further embodiment, the aqueous solution has a pH that is in the range from 6.0 to 7.

In particular, the stabilizing effect obtained is independent from the ionic strength of the aqueous solution of the therapeutically effective proteins/peptides, more particularly within at least one of the ionic strength range as defined below.

In a further embodiment, the aqueous solution has an ionic strength that is in the range from 0 to 300 mM.

In a further embodiment, the aqueous solution has an ionic strength that is in the range from 0 to 200 mM.

In a further embodiment, the aqueous solution has an ionic strength that is in the range from 1 to 150 mM.

Moreover the active concentrations of the lauryldimethylamineoxide and/or of one of its amine oxide analogs are independent of the antibody-derived therapeutically active protein concentrations. Furthermore the active concentrations of the lauryldimethylamineoxide and/or of one of its amine oxide analogs can be lower than that of other surfactants which make them attractive for highly concentrated protein stabilization.

In one embodiment the invention relates to a method of providing storage stability to an aqueous pharmaceutical formulation of an antibody-derived therapeutically active protein such as antibody, nanobody or fusion protein comprising admixing under sterile conditions in an aqueous solution an antibody-derived therapeutically active protein and an amount effective to stabilize said antibody-derived therapeutically active protein of at least one lauryldimethylamineoxide and/or of one of its amine oxide analogs.

In another embodiment the invention relates to a method of increasing the shelf life of an aqueous pharmaceutical formulation of an antibody-derived therapeutically active protein such as antibody, nanobody or fusion protein comprising admixing under sterile conditions in an aqueous solution an antibody-derived therapeutically active protein and an amount effective to stabilize said antibody-derived therapeutically active protein of at least one lauryldimethylamineoxide and/or of one of its amine oxide analogs.

In particular, the “shelf life” means the physical stability, and more particularly the physical stability as measured according to the test disclosed in Example 2.

In particular embodiments, the invention therefore also relates to a method of increasing the physical stability of an aqueous pharmaceutical formulation of an antibody-derived therapeutically active protein such as antibody, nanobody or fusion protein comprising admixing under sterile conditions in an aqueous solution an antibody-derived therapeutically active protein and an amount effective to stabilize said antibody-derived therapeutically active protein of at least one lauryldimethylamineoxide and/or of one of its amine oxide analogs.

The present invention also relates to the method to stabilize an antibody-derived therapeutically active protein in aqueous solutions with one lauryldimethylamineoxide and/or of one of its amine oxide analogs and to the storage-stable pharmaceutical compositions obtained.

The invention also relates to the method to stabilize a stored, aqueous pharmaceutical formulation of an antibody-derived therapeutically active protein such as an antibody, a nanobody or a fusion protein with at least one lauryldimethylamineoxide and/or of one of its amine oxide analogs.

The invention also relates to a storage-stable pharmaceutical composition, comprising an aqueous solution of:
at least an antibody-derived therapeutically active protein chosen amongst antibody, nanobody or fusion protein;
an amount effective to stabilize said antibody-derived therapeutically active protein of at least one lauryldimethylamineoxide and/or of one of its amine oxide analogs.

The stable liquid formulations of antibody-derived therapeutically active protein exhibit stability, low to undetectable levels of antibody fragmentation and/or aggregation, and very little to no loss of the biological activities of the antibodies (including antibody fragments thereof) during manufacture, preparation, transportation, and storage. Stability can be assessed by, for example, visual inspection, or size exclusion chromatography (SEC), in particular high performance size exclusion chromatography (HPSEC). The stability of the antibody formulations may be measured by, for example, high performance size exclusion chromatography (HPSEC). The techniques of static or dynamic light scattering (SLS or DLS), Fourier Transform Infrared Spectroscopy (FTIR), circular dichroism (CD), intrinsic tryptophan fluorescence, differential scanning calorimetry, light obscuration methods, and/or I-anilino-8-naphthalenesulfonic acid (ANS) protein binding techniques are also used to assess the phase behaviors, other physical properties and stability of the antibody molecules. Visual or spectroscopic inspection of aggregation is a preferred manner of determining stability. More particularly, the stability is measured by visual inspection, size exclusion chromatography (SEC), light obscuration methods, and/or dynamic light scattering (DLS). The choice of the method depends on the size of the particles.

The term “pharmaceutical formulation” refers to a preparation which is in such form as to permit the biological activity of the active ingredient to be effective, and which contains additional components, i.e excipients which are pharmaceutically acceptable.

A “storage-stable”, “stable” or “stabilized” formulation is one in which the antibody-derived therapeutically active protein such as antibody, nanobody or fusion protein retains its physical and/or chemical stability upon storage. Stability can be measured at a selected temperature for a selected time period. The aggregation during storage is used as an indicator of protein stability.

Physical and mechanical stress assays have been performed to simulate long term storage. Indeed, assays under accelerated and stress conditions provide product stability information for future product development as specified by the European Medicines Agency (ICH Topic Q 5 C Quality of Biotechnological Products: Stability Testing of Biotechnological/Biological Products, Note for Guidance on Quality of Biotechnological Products : Stability Testing of Biotechnological Products CPMP/ICH/138/95, July 1996).

Thus, a “stable” formulation may be one wherein less than 10% and preferably less than 5%, still more preferably less than 1% of the protein is present as an aggregate in the formulation.

The “stable” formulations of the invention retain biological activity under given manufacture, preparation, transportation and storage conditions. The antibody formulations of the invention maintain pharmaceutically acceptable aggregation profiles upon storage, for example, for extended periods (for example, but not limited to 6 months, 1 year, 2 years, 3 years or 5 years) at 2-8° C.

The antibody formulations of the invention may maintain pharmaceutically acceptable aggregation profiles upon storage, for example, for extended periods (for example, but not limited to 6 months, 1 year, 2 years, 3 years or 5 years) at room temperature, or even for periods (such as, but not limited to 1 week, 2 weeks, 3 weeks, 1 month, 2 months, 3 months, 6 months or 1 year) at elevated temperatures such as 38° C.-42° C.

In one aspect of the invention, the method or use provides an antibody-derived therapeutically active protein formulation with low to undetectable levels of aggregation. The phrase “low to undetectable levels of aggregation” as used herein refers to samples containing no more than 5%, no more than 4%, no more than 3%, no more than 2%, no more than 1% and no more than 0.5% aggregation by weight of protein as measured by visual inspection, size exclusion chromatography (SEC) in particular high performance size exclusion chromatography (HPSEC), static or dynamic light scattering (SLS or DLS), Fourier Transform Infrared Spectroscopy (FTIR), circular dichroism (CD). intrinsic tryptophan fluorescence, differential scanning calorimetry, light obscuration methods and/or ANS protein binding techniques. Furthermore, liquid formulations of the present invention exhibit almost no loss in biological activities of the antibody (including antibody fragment thereof) during the prolonged storage under the condition described above, as assessed by various immunological assays including, for example, enzyme-linked immunosorbent assay (ELISA) and radioimmunoassay to measure the ability of the antibody (including antibody fragment thereof) to immunospecifically bind to an antigen. The liquid formulations of the present invention retain after the storage for the above-defined periods more than 80%, more than 85%, more than 90%, more than 95%, more than 98%, more than 99%, or more than 99.5% of the initial biological activities.

The term “aqueous solution” refers to a solution in which water is the dissolving medium or solvent. When a substance dissolves in a liquid, the mixture is termed a solution. The dissolved substance i.e. the antibody-derived therapeutically active protein such as antibody, nanobody or fusion protein is the solute, and the liquid that does the dissolving (in this case water) is the solvent.

In a specific embodiment the antibody-derived therapeutically active protein is an antibody.

As used herein, the term “antibody” refers to an immunoglobulin molecule that recognizes and specifically binds to a therapeutic target, such as a protein, polypeptide, peptide, carbohydrate, polynucleotide, lipid, or combinations of the foregoing. The term “antibody” is referred to herein to encompass full-length monoclonal or polyclonal antibodies, as well as antibody fragments, such as Fab, Fab′, F(ab′)₂, and Fv fragments, single chain Fv (scFv) mutants and Fc fusion proteins. Therapeutic antibodies of the invention include multispecific antibodies such as bispecific antibodies generated from at least two full-length antibodies, chimeric antibodies, humanized antibodies, human antibodies, fusion proteins, peptibodies comprising an antigen recognition portion or an Fc portion of an antibody, and modified immunoglobulin molecules comprising an antigen recognition site (or portion thereof) or Fc domain, so long as the protein exhibits the desired biological (e.g., therapeutic) activity. A therapeutic antibody can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Therapeutic antibodies can be naked or conjugated to other molecules such as toxins, radioisotopes, etc.

The method, use or composition according to the invention are further discribed by the following embodiments.

In a specific embodiment the antibody is a monoclonal antibody.

In a specific embodiment the antibody is a polyclonal antibody.

In a specific embodiment the antibody is a bispecific antibody.

In a specific embodiment the antibody is an IgG.

In a specific embodiment the antibody is an IgA.

In a specific embodiment the antibody is an IgE.

In a specific embodiment the antibody is an IgD.

In a specific embodiment the antibody is an IgM.

In a specific embodiment the antibody is an IgG1.

In a specific embodiment the antibody is an IgG2.

In a specific embodiment the antibody is an IgG3.

In a specific embodiment the antibody is an IgG4.

In a specific embodiment the antibody is an IgA1.

In a specific embodiment the antibody is an IgA2.

The term “antibody fragment” refers to a portion of an antibody that includes an antigen recognition site (or portion thereof) and/or non-antigen recognition site, such as an effector domain, as well as variants or derivatives of an antibody. Thus, “antibodies” include polypeptides containing an antibody fragment that has an effector domain and/or all or a portion of an antigen recognition site. Antibody fragments include single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VII domain, fragments produced by a Fab expression library, anti-idiotypic (anti-Id) antibodies, and Fc fusion proteins. Antibody fragments can be of any type (e.g., IgG, IgE, IgM, IgD, IgA, and IgY), class (e.g., IgG1, IgG2, IgG3, IgG4, IgA1 and IgA2) or subclass of immunoglobulin molecule. Antibody fragments, including single-chain antibodies, may comprise one or more CDR regions alone (e.g., CDR2 and CDR3) or any combination of CDR regions, the variable region(s) alone or in combination with the entirety or a portion of the following: hinge region, CH1, CH2, and CH3 domains. Antibody fragments can contain any combination of variable region(s) with a hinge region, CH1, CH2, and CH3 domains. Antibody fragments may additionally, or alternatively, include portions of an antibody constant region that confer antibody effector function (e.g., immunoglobulin effector domain sequences) or that mediate antibody half-life. In particular embodiments, therapeutic antibodies of the invention comprise an Fc domain. In additional embodiments, the antibodies are Fc fusion proteins. Antibody fragments can be from any appropriate source and may be of synthetic (e.g., chimeric, humanized and otherwise modified antibodies) or animal origin (e.g., birds and mammals). Antibody fragments that recognize specific epitopes and/or that compete for target binding with another antibody or protein can be generated, identified, and characterized using techniques known in the art.

The terms “therapeutic antibody” and “therapeutically effective antibody” are used interchangeably herein and refer to an antibody that when administered to a subject in a sufficiently effective amount, prevents, delays, alleviates or arrests the onset and/or further development of the symptoms, complications, or biochemical indicia of a disease, condition, or disorder in the subject (e.g., a patient such as a human patient, non-human primate, or an experimental animal such as a rabbit, rat, mouse or other animal).

The terms “compete,” “compete for binding” and “competes with” are relative terms used to describe an antibody that produces a 50% inhibition of binding to a target by an antibody or reference cognate ligand as determined in a standard competition assay as described herein or otherwise known in the art, including, but not limited to, competitive assay systems using techniques such as radioimmunoassays (RIA), enzyme immunoassays (ETA), preferably the enzyme linked immunosorbent assay (ELISA), “sandwich” immunoassays, immunoradiometric assays, fluorescent immunoassays, luminescent, electrochemical luminescent, and immunoelectrophoresis assays. Methods for determining binding and affinity of candidate binding molecules are known in the art and include, but are not limited to, affinity chromatography, size exclusion chromatography, equilibrium dialysis, fluorescent probe displacement, and plasma resonance.

An antibody is said to “competitively inhibit” binding of a reference molecule to a given epitope if it binds to that epitope to the extent that it blocks, to some degree, binding of the reference molecule to the epitope. Competitive inhibition can be determined by any method known in the art, for example, competition ELISA assays. As used herein, an antibody can be said to competitively inhibit binding of the reference molecule to a given epitope, for example, by at least 90%, at least 80%, at least 70%, at least 60%, or at least 50%.

In some embodiments, the therapeutically active antibody is a member selected from: Muromonab-CD3 (product marketed with the brand name Orthoclone Okt3®), Abciximab (product marketed with the brand name Reopro®), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), I'Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-I131 (product marketed with the brand name Bexxar®), Efalizumab (product marketed with the brand name Raptiva®), Cetuximab (product marketed with the brand name Erbituxn®), I′Ibritumomab tiuxetan (product marketed with the brand name Zevalin®), I′Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), I'Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), I'Ustekinumab (product marketed with the brand name Stelara®), Tocillzumab (product marketed with the brand name RoActemra®, Actemra®), I'Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), and Pertuzumab (product marketed with the brand name Perjeta®).

In a specific embodiment, the antibody-derived therapeutically active protein is a fusion protein.

As used herein, the term “fusion proteins” by itself or as part of another phrase, refers to proteins created through the joining of two or more genes which originally coded for separate proteins/peptides. Translation of this fusion gene results In a single polypeptide with functional properties derived from each of the original proteins, Recombinant fusion proteins are created artificially by recombinant DNA technology for use in biological research or therapeutics. Chimeric mutant proteins occur naturally when a complex mutation, such as a chromosomal translocation, tandem duplication, or retrotransposition creates a novel coding sequence containing parts of the coding sequences from two different genes. Naturally occurring fusion proteins are commonly found in cancer cells, where they may function as oncoproteins.

In one embodiment, the therapeutically active fusion protein is a member selected from: Abatacept (product marketed with the brand name Orencla®), Etanercept (product marketed with the brand name Enbrel®), Rilonacept (product marketed with the brand name Arcalyst®) and Alefacept (product marketed with the brand name Amevive®),

In a specific embodiment the antibody-derived therapeutically active protein Is a nanobody.

As used herein, the term “nanobody” or “single domain antibody” refers to an antibody fragment consisting of a single monomeric variable antibody domain. Like a whole antibody, it is able to bind selectively to a specific antigen. Nanobodies are described In C. Vincke and S. Muyldermans, In D. Saerens and S. Muyldermans (eds.) Single Domain Antibodies: Methods and Protocols, Methods in Molecular Biology, vol. 911; and Med Microbial Immunol, (2009).

By “amine oxide analogs of lauryldimethylamineoxide” it is meant amine oxide chosen amongst the amine oxides of formula I:

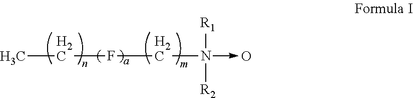

wherein
m represents an integer comprised in the interval from 0 to 17, 0≤m≤17,
n represents an integer comprised in the interval from 0 to 17, 0≤n≤17,
a represents an integer equal to 0 or 1,
9≤n+m≤17,
If m=0, then a=0,
F represents a function chosen in the group constituted by the functions amide, ester, carbamate and urea,
R1 and R2, identical or different, represent alkyl chains comprising from 1 to 4 carbon atoms.

According to the IUPAC definition (PAC, 1995, 67, 1307) the aminoxides of formula I can also be represented such as in formula I′:

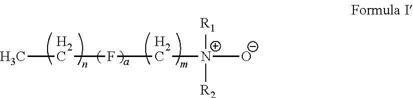

In a further embodiment, the analogs of lauryldimethylamineoxide are chosen amongst the amine oxide analogs of formula I, wherein n represents an integer comprised in the interval from 9 to 13, 9≤n≤13.

In a further embodiment, the analogs of lauryldimethylamineoxide are chosen amongst the amine oxide analogs of formula I, wherein a=0 and m=0 and the amine oxide is chosen amongst the compounds of formula

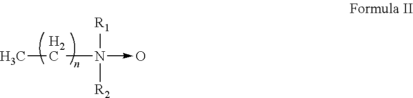

wherein,
n represents an integer comprised in the interval from 9 to 17, 9≤n≤17,
R1 and R2, identical or different, represent alkyl chains comprising from 1 to 4 carbon atoms.

According to the IUPAC definition (PAC, 1995, 67, 1307) the aminoxides of formula H can also be represented such as in formula

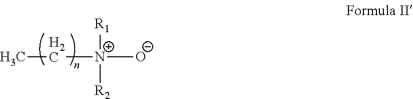

In a further embodiment, the analogs of lauryldimethylamineoxide are chosen amongst the amine oxide analogs of formula II, wherein n represents an integer comprised in the interval from 9 to 13, 9≤n≤13.

In a further embodiment, the concentration of the at least one lauryldimethylamineoxide and/or amine oxide analogs is in the range from 0.01 mM to 100 mM.

In a further embodiment, the concentration of the at least one lauryldimethylamineoxide and/or amine oxide analogs is in the range from 0.1 mM to 10 mM.

In a further embodiment, the concentration of the at least one lauryldimethylamineoxide and/or amine oxide analogs is in the range from 0.1 mM to 5 mM.

In a further embodiment, the concentration of the at least one lauryldimethylamineoxide and/or amine oxide analogs is in the range from 0.2 mM to 2 mM.

In a further embodiment, the concentration of the at least one lauryldimethylamineoxide and/or amine oxide analogs is in the range from 0.5 mM to 1.5 mM.

In a further embodiment, the at least one amine oxide analog is N,N-Dirnethyldecylamine N-oxide (CAS 2605-79-0).

In a further embodiment, the at least one amine oxide analog is N,N-Lauryldimethylamine N-oxide (CAS 1643-20-5).

In a further embodiment, the at least one amine oxide analog is N,N-Dimethyitetradecylamine N-oxide (CAS 3332-27-2).

In a further embodiment, the at least amine oxide analog is N-2-(dimethylnitroryl)ethyldodecanamide (CAS 86321-42-8).

In a further embodiment, the concentration of the antibody-derived therapeutically active protein is in the range from 1 to 350 mg/mL.

In a further embodiment, the concentration of the antibody-derived therapeutically active protein is in the range from 1 to 250 mg/mL.

In a further embodiment, the concentration of the antibody-derived therapeutically active protein is in the range from 1 to 150 mg/mL.

In a further embodiment, the concentration of the antibody-derived therapeutically active protein is in the range from 1 to 100 mg/mL.

In a further embodiment, the concentration of the antibody-derived therapeutically active protein is in the range from 1 to 75 mg/mL.

In a further embodiment, the concentration of the antibody-derived therapeutically active protein is in the range from 1 to 50 mg/mL.

In a further embodiment, the concentration of the antibody-derived therapeutically active protein is in the range from 50 to 350 mg/mL.

In a further embodiment, the concentration of the antibody-derived therapeutically active protein is in the range from 50 to 250 mg/mL.

In a further embodiment, the concentration of the antibody-derived therapeutically active protein is in the range from 50 to 150 mg/mL.

In a further embodiment, the concentration of the antibody-derived therapeutically active protein is in the range from 50 to 100 mg/mL.

In a further embodiment, the concentration of the antibody-derived therapeutically active protein is in the range from 5 to 75 mg/mL.

In a further embodiment, the concentration of the antibody-derived therapeutically active protein is in the range from 5 to 25 mg/mL.

In a further embodiment, the composition has a pH that is in the range from 5.0 to 8.0.

In a further embodiment, the composition has a pH that is in the range from 5.5 to 7.8.

In a further embodiment, the composition has a pH that is in the range from 5 to 6.5.

In a further embodiment, the composition has a pH that is in the range from 5.5 to 6.5.

In a further embodiment, the composition has a pH that is in the range from 6 to 8.

In a further embodiment, the composition has a pH that is in the range from 6 and 7.5.

In a further embodiment, the composition has a pH that is in the range from 6 to 7.

In a further embodiment, the composition has an osmolality that is in the range from 200 to 600 mOsm/kg.

In a further embodiment, the composition has an osmolality that is in the range from 200 to 500 mOsm/kg.

In a further embodiment, the composition has an osmolality that is in the range from 200 to 400 mOsm/kg.

In one embodiment at least one additional surfactant is added to the composition.

“Surfactants” are surface active agents that can exert their effect at surfaces of solid-solid, solid-liquid, liquid-liquid, and liquid-air because of their chemical composition, containing both hydrophilic and hydrophobic groups.

In one embodiment of the invention, the at least one additional surfactant is chosen amongst polysorbate 20 (CAS 9005-64-5) and polysorbate 80 (CAS 9005-65-6), in particular is polysorbate 20.

In another embodiment of the invention, the at least one additional surfactant is chosen amongst n-Dodecyl β-D-maltoside (CAS 69227-93-6), Polyoxyl 35 hydrogenated castor oil (Cremophor EL, CAS 61791-12-6), and Polyoxyethylene-polyoxypropylene block copolymer (Pluronic F-68, CAS 9003-11-6).

In one embodiment the surfactant is a zwitterionic surfactant i.e. (Lauryldimethylammonio)acetate (S6, CAS 683-10-3, Sigma-Aldrich).

In another embodiment the compositions comprises only one lauryldimethylamineoxide and/or of one of its amine oxide analogs as surfactant.

In one embodiment at least one additional viscosity reducer is added to the composition.

A “viscosity reducer” is a compound that is capable of measurably reducing viscosity of an aqueous protein-containing formulation. In one embodiment the viscosity reducer is arginine hydrochloride or one compound chosen amongst the compounds described in U.S. provisional application in the name of Adocia No. 61/68,2003.

In one embodiment at least one pharmaceutically acceptable acid is added to the composition.

A “pharmaceutically acceptable acid” includes inorganic and organic acids which are non toxic at the concentration and manner in which they are formulated. For example, suitable acids include hydrochloric, phosphoric, citric, acetic, ascorbic, ethylenediaminetetracetic acid (EDTA) and tartaric acids.

In one embodiment at least one pharmaceutically acceptable base is added to the composition.

“Pharmaceutically-acceptable bases” include inorganic and organic bases which are non-toxic at the concentration and manner in which they are formulated. For example, suitable bases include inorganic bases formed from metals such as sodium, potassium, magnesium and calcium.

For example, suitable bases include NaOH and KOH.

Additional pharmaceutically acceptable inorganic bases useable with the present invention include ammonium hydroxide.

Additional pharmaceutically acceptable acids and bases useable with the present invention include those which are derived from the amino acids, for example, histidine, arginine, glycine, phenylalanine, lysine and asparagine.

In one embodiment at least one buffer is added to the composition.

“Buffers” and salts include those derived from both acid and base addition salts of the above indicated acids and bases.

In one embodiment at least one pharmaceutical diluent is added to the composition.

For example, suitable diluents include injection sterilized water, buffer water solution and a NaCl solution (NaCl 0.9%).

In one embodiment at least one pharmaceutical preservative is added to the composition.

In particular embodiments, the lauryldimethylamineoxide and/or amine oxide analog(s) are at a concentration ≥ to 2 times its CMC (Critical Micelle Concentration).

In particular embodiments, the lauryldimethylamineoxide and/or amine oxide analog(s) are at a concentration to 4 times its CMC.

In particular embodiments, the lauryldimethylamineoxide and/or amine oxide analog(s) are at a concentration ≥to 6 times its CMC.

In particular embodiments, the lauryldimethylamineoxide and/or amine oxide analog(s) are at a concentration to 8 times its CMC.

In particular embodiments, the lauryldimethylamineoxide and/or amine oxide analog(s) are at a concentration between its CMC and 10 times its CMC.

In particular embodiments, the lauryldimethylamineoxide and/or amine oxide analog(s) are at a concentration between its CMC and 5 times its CMC.

In particular embodiments, the lauryldimethylamineoxide and/or amine oxide analog(s) are at a concentration that is ≥10 times its CMC.

In particular embodiments, the lauryldimethylamineoxide and/or amine oxide analog(s) are at a concentration that is ≥5 times its CMC.

The invention provides for pharmaceutical compositions for intravenous administration comprising at least 1 mg/mL of an antibody-derived therapeutically active protein.

The invention provides for pharmaceutical compositions for intravenous administration comprising from 1 mg/mL to 50 mg/mL of an antibody-derived therapeutically active protein.

In additional embodiments, the invention provides for pharmaceutical compositions for subcutaneous administration comprising at least 50 mg/mL of a therapeutic antibody, nanobody or fusion protein.

In additional embodiments, the invention provides for pharmaceutical compositions for subcutaneous administration comprising from 50 mg/mL to 350 mg/mL of a therapeutic antibody, nanobody or fusion protein.

In additional embodiments, the invention provides for pharmaceutical compositions for subcutaneous administration comprising from 50 mg/mL to 250 mg/mL of a therapeutic antibody, nanobody or fusion protein.

In additional embodiments, the invention provides for pharmaceutical compositions for subcutaneous administration comprising from 50 mg/mL to 150 mg/mL of a therapeutic antibody, nanobody or fusion protein.

In additional embodiments, the invention provides for pharmaceutical compositions for subcutaneous administration comprising from 50 mg/mL to 100 mg/mL of a therapeutic antibody, nanobody or fusion protein.

According to some embodiments the invention encompasses a method of administering a formulation of the invention to a patient comprising the steps of injecting an aqueous formulation that comprises at least an intravenous injection, subcutaneous injection or intramuscular injection.

The invention also concerns a pharmaceutical container, comprising a hermetically sealed vessel and the pharmaceutical composition as described above.

In one embodiment the invention, the pharmaceutical container, where in the vessel is a vial, bottle, pre-filled syringe or pre-filled auto-injector.

### EXAMPLE 1

### Solution of Bevacizumab (Avastin®) at 10 mg/mL

The formulation at 25 mg/mL (pH 6.2) is marketed by Genentech/Hoffmann-La Roche. For the purpose of the experiment, this formulation was diluted to 10 mg/mL and was completed with its excipients in order to reach the following concentrations in the final solution: 158 mM of trehalose, 50 mM of sodium phosphate and 0.33 mM of polysorbate 20.

### EXAMPLE 2

### Solution of Infliximab (Remicad®) at 5 mg/mL

The formulation is a freeze-dried product reconstituted at 10 mg/mL with water (pH 7.2). In Europe, it is marketed by Schering-Plough. For the purpose of the experiment, this formulation was diluted to 5 mg/mL and was completed with its excipients in order to reach the following concentrations in the final solution: 146 mM of saccharose, 50 mM of sodium phosphate and 38 μM of polysorbate 80.

### EXAMPLE 3

### Solution of Trastuzumab (Herceptin®) at 10 mg/mL

The formulation is a freeze-dried product reconstituted at 22 mg/mL with water for injection (pH 6). In Europe, it is marketed by Hoffmann-La Roche. For the purpose of the experiment, this formulation was diluted to 10 mg/mL and was completed with its excipients in order to reach the following concentrations in the final solution: 2.3 mM of L-histidine, 52.8 mM of trehalose and 72 μm of polysorbate 20.

### EXAMPLE 4

### Solution of Rituximab (MabThera® or Rituxan®) at 10 mg/mL

The formulation at 10 mg/mL is marketed by Hoffmann-La Roche in Europe (pH 6.5). This formulation contains 153 mM of sodium chloride, 25 mM of sodium citrate and 534 μM of polysorbate 80. The commercial formulation was used as such.

### EXAMPLE 5

### Solution of Cetuximab (Erbitux®) at 75 mg/mL

The formulation at 5 mg/mL is marketed by Merck (pH 5.3-5.7) in Europe. This formulation contains sodium chloride, glycine, citric acid and polysorbate 80 in unknown amounts. For the purpose of the experiment, it was concentrated at 75 mg/mL.

### EXAMPLE 6

### Solution of Abatacept (Orencia®) at 25 mg/mL

The formulation is a freeze-dried product reconstituted at 25 mg/mL with water for injection (pH 7.2-7.8). It is marketed by Bristol-Meyer Squibb Pharma in Europe. Abatacept is a fusion protein composed of the Fc region of the immunoglobulin IgG1 fused to the extracellular domain of CTLA-4. This solution contains 50 mg/mL of maltose, 1.7 mg/mL of sodium phosphate and 1.5 mg/mL of sodium chloride. The commercial formulation was used as such.

### EXAMPLE 7

### Stabilizing Effect on Monoclonal Antibody Formulations

The physical stability of monoclonal antibody formulations is evaluated using the following mechanical stress: 0.5 mL of the formulation is shaken on a wheel agitator at a speed of 80 rpm in presence of 2 glass beads of 2 mm-diameter.

For each monoclonal antibody, three conditions of formulation were tested:

1. The first formulation is the control formulation described in example 1-5.

2. The second formulation is the formulation described in example 1-5 to which polysorbate is added. The same kind of polysorbate as the commercial formulation is added (e.g. for Bevacizumab: polysorbate 20, for Infliximab: polysorbate 80). The final concentration of polysorbate is equal to 1.5 mM.

3. The last formulation is the formulation described in example 1-5 to which an amine oxide compound is added. The concentration of the amine oxide in the formulation is equal to 1.5 times its CMC.

Monitoring of these experiments is done by visual inspection and light scattering measurements.

Alkyl amine oxide compounds tested include N,N-Dimethyldecylamine N-oxide (Amine oxide 1, CAS 2605-79-0, Sigma-Aldrich, CMC=10 mM), N,N-Lauryldimethylamine N-oxide (Amine oxide 2, CAS 1643-20-5, Sigma-Aldrich, CMC=1 mM), N,N-Dimethyltetradecylamine N-oxide (Amine oxide 3, CAS 3332-27-2, Sigma-Aldrich, CMC=0.14 mM) and N-2-(dimethylnitroryl)ethyldodecanamide (Amine oxide 4, CAS 86321-42-8, Sigma-Aldrich, CMC=1.8 mM).

All these amine oxide compounds are employed at a concentration equal to 1.5 times their CMC.

In all the tables: “Clear” means stable , and “Turbid” means unstable.

The table 1 below indicates the concentration, the buffer composition, the pH and the ionic strength of the aqueous solutions of monoclonal antibodies (commercial solutions).

TABLE 1

| Monoclonal | | | |
|---|---|---|---|
| antibody | Concentration | Buffer Composition | pH |
| Bevacizumab | 10 | 50 mM sodium phosphate - | 6.2 |
| | | 158 mM trehalose - | |
| | | 0.33 mM polysorbate 20 | |
| Infliximab | 5 | 50 mM sodium phosphate - | 7.2 |
| | | 146 mM saccharose - | |
| | | 0.038 mM polysorbate 80 | |
| Trastuzumab | 10 | 2.3 mM L-histidine - | 6.0 |
| | | 52.8 mM trehalose - | |
| | | 0.072 mM polysorbate 20 | |
| Rituximab | 10 | 25 mM sodium citrate - | 6.5 |
| | | 153 mM sodium chloride - | |
| | | 0.534 mM polysorbate 80 | |
| Abatacept | 25 | 14 mM sodium phosphate - | 7.2-7.8 |
| | | 25 mM sodium chloride - | |
| | | 146 mM maltose | |

The table 2 below indicates the visual aspect of each formulation at the end of the stress test.

TABLE 2

| | | | | | | | pH | Ionic strength | |
|---|---|---|---|---|---|---|---|---|---|
| | | Control | Control | Control | Control | Control | (for all of the | (for of all the | |
| | | formulation + | formulation + | formulation + | formulation + | formulation + | formulations | formulations | |
| Monoclonal | Control | added | added amine | added amine | added amine | added amine | presently | presently | |
| antibody | formulation | polysorbate | oxide 1 | oxide 2 | oxide 3 | oxide 4 | tested) | tested) | |
| Bevacizumab | Turbid | Turbid | Clear | Clear | Clear | Clear | 6.2 | 66 | mM |
| Infliximab | Turbid | Turbid | Clear | Clear | Clear | Clear | 7.2 | 119 | mM |
| Trastuzumab | Turbid | Turbid | Clear | Clear | Clear | Clear | 6.0 | 2.3 | mM |
| Rituximab | Turbid | Turbid | Not tested | Clear | Not tested | Not tested | 6.5 | 291 | mM |

| | | | | | | | | |
|---|---|---|---|---|---|---|---|---|
| Cetuximab | Turbid | Turbid | Not tested | Clear | Clear | Not tested | 5.3-5.7 | — |

In view of the above results, it could be concluded that physical stability of the five monoclonal antibody formulations evaluated is drastically increased in presence of amine oxide compounds.

The stabilizing effect obtained is independent from the pH and from the ionic strength of the aqueous solution of the therapeutic antibody-derived therapeutically active protein.

### EXAMPLE 8

### Stabilizing Effect on a Fusion Protein

The physical stability of formulations containing a fusion protein was evaluated using the following mechanical stress: 0.5 mL of the formulation was shaken on a wheel agitator at a speed of 80 rpm in presence of 2 glass beads of 2 mm-diameter.

Two formulations were tested:

1. The first formulation is the control formulation described in example 6.

2. The second formulation is the control formulation described in example 6 to which amine oxide 2 is added. The concentration of amine oxide 2 in the formulation is 1.5 mM which corresponds to 1.5 times its CMC.

Monitoring of this experiment is done by visual inspection and light scattering measurements.

The table 3 below indicates the visual aspect of each formulation at the end of the stress test.

TABLE 3

| Fusion | Control | Control formulation + |
|---|---|---|
| protein | formulation | Amine oxide 2 |
| Abatacept | Turbid | Clear |

In view of the above results, it could be concluded that the physical stability of the fusion protein formulation evaluated is drastically increased in presence of alkyl amine oxide surfactant,

### EXAMPLE 9

### Comparison of the Stabilizing Effect of Different Surfactants

The physical stability of different formulations containing Bevacizumab and various surfactants is evaluated using the following mechanical stress: 0.5 mL of the formulation is shaken on a wheel agitator at a speed of 80 rpm in presence of 2 glass beads of 2 mm-diameter.
The first formulation is the control formulation described in example 1.
The others formulations consist in the control formulation described in example 1 to which a surfactant is added.

Monitoring of this experiment is done by visual inspection and light scattering measurements.

Non-ionic surfactants tested include polysorbate 20 (S1, CAS 9005-64-5, InResa Pharma), polysorbate 80 (S2, CAS 9005-65-6, Sigma-Aldrich), n-Dodecyl β-D-maltoside (53, CAS 69227-93-6, Sigma-Aldrich), Polyoxyl 35 hydrogenated castor oil (S4, Cremophor EL, CAS 61791-12-6, Sigma-Aldrich), Polyoxyethylene-polyoxypropylene block copolymer (S5, Pluronic F-68, CAS 9003-11-6, Sigma-Aldrich).

Zwitterionic surfactant tested is (Lauryldimethylammonio)acetate (S6, CAS 683-10-3, Sigma-Aldrich).

Cationic surfactants tested are Hexadecyltrimethylammonium bromide (S7, CAS 57-09-0, Sigma-Aldrich) and Dodecyltrimethylammonium bromide (S8, CAS 1119-94-4, Sigma-Aldrich).

Oxide surfactants tested are Dimethyldecylphosphine oxide (S9, CAS 2190-95-6, Sigma-Aldrich) and Dodecylmethyl sulfoxides (S10, CAS 3079-30-9, Sigma-Aldrich).

Alkyl amine oxide compounds tested are N,N-Dimethyldecylamine N-oxide (Amine oxide 1, CAS 2605-79-0, Sigma-Aldrich, CMC=10 mM), Lauryldimethylamine N-oxide (Amine oxide 2, CAS 1643-20-5, Sigma-Aldrich, CMC=1 mM), N,N-Dimethyltetradecylamine N-oxide (Amine oxide 3, CAS 3332-27-2, Sigma-Aldrich, CMC=0.14 mM) and N-[2-(dimethylnitroryl)ethyldodecanamide (Amine oxide 4, CAS 86321-42-8, Sigma-Aldrich, CMC=1.8 mM).

All theses surfactants are employed at a concentration equal to 1.5 times their CMC.

The table 4 below indicates the visual aspect of each formulation at the end of the stress test.

TABLE 4

| Formulation | Visual observations |
|---|---|
| Control formulation | Turbid |
| Control formulation + S1 | Turbid |
| Control formulation + S2 | Turbid |
| Control formulation + S3 | Turbid |
| Control formulation + S4 | Turbid |
| Control formulation + S5 | Turbid |
| Control formulation + S6 | Turbid |
| Control formulation + S7 | Turbid |
| Control formulation + S8 | Turbid |
| Control formulation + S9 | Turbid |
| Control formulation + S10 | Turbid |
| Control formulation + Amine oxide 1 | Clear |
| Control formulation + Amine oxide 2 | Clear |
| Control formulation + Amine oxide 3 | Clear |
| Control formulation + Amine oxide 4 | Clear |

Control and surfactants S1 to S10 lead to formulations having a turbid aspect contrary to amine oxide containing formulations that are still clear at the end of the stress test. So it is clearly demonstrated that S1 to S10 surfactants are not able to stabilize Bevacizumab formulations, but in the same conditions the amine oxide compounds are able to increase the Bevacizumab formulations stability.

### EXAMPLE 10

### Pharmaceutical Formulations

The following examples are increased stability pharmaceutical formulations obtained following the above described invention.

| Ingredients: | Concentration (mg/mL): |
|---|---|
| Infliximab | 10 |
| Sodium phosphate (monobasic, monohydrate) | 0.22 |
| Sodium Phosphate (dibasic, dihydrate) | 0.61 |
| Sucrose | 50 |
| N,N-Lauryldimethylamine N-oxide | 0.3 |
| Water for injection, USP | qs |
| Infliximab | 10 |
| Sodium phosphate (monobasic, monohydrate) | 0.22 |
| Sodium Phosphate (dibasic, dihydrate) | 0.61 |
| Sucrose | 50 |
| N,N-Dimethyltetradecylamine N-oxide | 0.04 |
| Water for injection, USP | qs |
| Bevacizumab | 25 |
| Sodium phosphate (monobasic, monohydrate) | 5.8 |
| Sodium Phosphate (dibasic, anhydrous) | 1.2 |
| α,α-trehalose dihydrate | 60 |
| N,N-Dimethyldecylamine N-oxide | 0.3 |
| Water for injection, USP | qs |
| Bevacizumab | 25 |
| Sodium phosphate (monobasic, monohydrate) | 5.8 |
| Sodium Phosphate (dibasic, anhydrous) | 1.2 |
| α,α-trehalose dihydrate | 60 |
| N,N-Dimethyltetradecylamine N-oxide | 0.04 |
| Water for injection, USP | qs |
| Abatacept | 25 |
| Sodium phosphate (monobasic) | 1.72 |
| Maltose | 50 |
| Sodium Chloride | 1.46 |
| N,N-Lauryldimethylamine N-oxide | 0.3 |
| Water for injection, USP | qs |
| Abatacept | 25 |
| Sodium phosphate (monobasic) | 1.72 |
| Maltose | 50 |
| Sodium Chloride | 1.46 |
| N,N-Dimethyltetradecylamine N-oxide | 0.04 |
| Water for injection, USP | qs |
| Trastuzumab | 22 |
| L-Histidine HCl | 0.49 |
| L-Histidine | 0.32 |
| α,α-trehalose dihydrate | 20 |
| N,N-Lauryldimethylamine N-oxide | 0.3 |
| Water for injection, USP | qs |
| Trastuzumab | 22 |
| L-Histidine HCl | 0.49 |
| L-Histidine | 0.32 |
| α,α-trehalose dihydrate | 20 |
| N,N-Dimethyltetradecylamine N-oxide | 0.04 |
| Water for injection, USP | qs |
| Rituximab | 10 |
| Sodium citrate dihydrate | 7.35 |
| Sodium chloride | 9 |
| N,N-Lauryldimethylamine N-oxide | 0.3 |
| Water for injection, USP | qs |

qs means Quantum Satis

1. A storage-stable pharmaceutical composition, comprising an aqueous solution of:
at least an antibody-derived therapeutically active protein chosen amongst antibody, nanobody or fusion protein;
an amount effective to stabilize said antibody-derived therapeutically active protein of at least one lauryldimethylamineoxide and/or of one of its amine oxide analogs.

2. The pharmaceutical composition of claim 1, wherein the analogs of lauryldimethylamineoxide are chosen amongst the amine oxide analogs of formula I ; 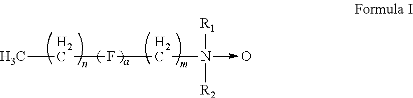
wherein,
m represents an integer comprised in the interval from 0 to 17, 0≤m≤17,
n represents an integer comprised in the interval from 0 to 17, 0≤n≤17,
a represents an integer equal to 0 or 1,
9<m+n≤17,
If m=0, then a=0
F represents a function chosen in the group constituted by the functions amide, ester, carbamate and urea,
R1 and R2, identical or different, represent alkyl chains comprising from 1 to 4 carbon atoms.

3. The pharmaceutical composition according to claim 2, wherein a=0 and m=0 and the amine oxide is chosen amongst the compounds of formula II: 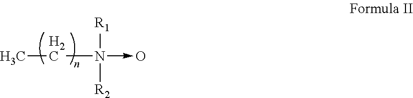
wherein,
n represents an integer comprised in the interval from 9 to 17, 9≤n≤17,
R1 and R2, identical or different, represent alkyl chains comprising from 1 to 4 carbon atoms.

4. The pharmaceutical composition according to claim 1, wherein the concentration of the at least one lauryldimethylamineoxide and/or amine oxide analogs is from 0.01 to 100 mM.

5. The pharmaceutical composition according to claim 1, wherein the concentration of the at least one lauryldimethylamineoxide and/or amine oxide analogs is from 0.1 to 10 mM.

6. The pharmaceutical composition according to claim 1, wherein the at least one amine oxide analogs is N,N-Dimethyldecylamine N-oxide (CAS 2605-79-0).

7. The pharmaceutical composition according to claim 1, wherein the at least one amine oxide analogs is N,N-Lauryldimethylamine N-oxide (CAS 1643-20-5).

8. The pharmaceutical composition according to claim 1, wherein the at least one amine oxide analogs is N,N-Dimethyltetradecylamine N-oxide (CAS 3332-27-2).

9. The pharmaceutical composition according to claim 1, wherein the at least one amine oxide analogs is N-2-(dimethylnitroyl)ethyldodecanamide (CAS 86321-42-8).

10. The pharmaceutical composition according to claim 1, further comprising an additional surfactant.

11. The pharmaceutical composition according to claim 1, wherein said composition has a pH that is in the range from 5 to 8.

12. The pharmaceutical composition according to claim 1, wherein said composition has a pH that is in the range from 5.5 to 7.8.

13. The pharmaceutical composition according to claim 1, wherein said composition has an osmolality in the range from 200 to 600 mOsm/kg.

14. The pharmaceutical composition according to claim 1, wherein said composition has an osmolality in the range from 200 to 500 mOsm/kg.

15. The pharmaceutical composition according to claim 1, wherein the antibody-derived therapeutically active protein is an antibody.

16. The pharmaceutical composition according to claim 1, wherein the antibody-derived therapeutically active protein is a nanobody.

17. The pharmaceutical composition according to claim 1, wherein the antibody-derived therapeutically active protein is a fusion protein.

18. The pharmaceutical composition according to claim 1, wherein the concentration of the antibody-derived therapeutically active protein is from 1 to 350 mg/mL.

19. A pharmaceutical container, comprising a hermetically sealed vessel and the pharmaceutical composition of claim 1.

20. The pharmaceutical container of claim 19, where in the vessel is a vial, bottle, pre-filled syringe or pre-filled auto-injector.

21. (canceled)

22. (canceled)

23. A method of providing storage stability to an aqueous pharmaceutical formulation of an antibody-derived therapeutically active protein, comprising admixing under sterile conditions in an aqueous solution an antibody-derived therapeutically active protein, and amount effective to stabilize said antibody-derived therapeutically active protein of at least one lauryldimethylamineoxide and/or of one of its amine oxide analogs.

24. Method according to claim 23, wherein the at least one amine oxide analog is chosen amongst the amine oxides of formula I: 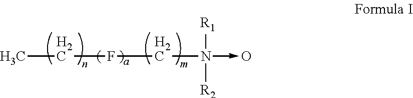
wherein
m represents an integer comprised in the interval from 0 to 17, 0≤m≤17,
n represents an integer comprised in the interval from 0 to 17, 0≤n≤17,
a represents an integer equal to 0 or 1,
9≤m+n≤17,
if m=0, then a=0
F represents a function chosen in the group constituted by the functions amide, ester, carbamate and urea,
R1 and R2, identical or different, represent alkyl chains comprising from 1 to 4 carbon atoms.

25. A method of increasing the shelf life of an aqueous pharmaceutical formulation of an antibody-derived therapeutically active protein, comprising admixing under sterile conditions in an aqueous solution an antibody-derived therapeutically active protein, and amount effective to stabilize said antibody-derived therapeutically active protein of at least one lauryldimethylamineoxide and/or of one of its amine oxide analogs.

26. A method according to claim 25, wherein the at least amine oxide analog is chosen amongst the amineoxides of formula I: 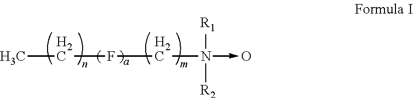
wherein
m represents an integer comprised in the interval from 0 to 17, 0≤m≤17,
n represents an integer comprised in the interval from 0 to 17, 0≤n≤17,
a represents an integer equal to 0 or 1,
9≤m+n≤17,
If m=0, then a=0
F represents a function chosen in the group constituted by the functions amide, ester, carbamate and urea,
R1 and R2, identical or different, represent alkyl chains comprising from 1 to 4 carbon atoms.
